(19) United States
(12) Patent Application Publication
(10) Pub. No.: US 2011/0200554 A1
(43) Pub. Date: Aug. 18, 2011

(54) THERAPEUTIC AGENT FOR HEPATITIS C

(76) Inventors: Akito Nozaki, Okayama (JP); Katsuaki Tanaka, Kanagawa (JP); Masanori Ikeda, Okayama (JP); Nobuyuki Kato, Okayama (JP)

(21) Appl. No.: 13/124,584

(22) PCT Filed: Oct. 16, 2009
(86) PCT No.: PCT/JP2009/067942

§ 371 Date: Apr. 15, 2011

(30) Foreign Application Priority Data
Oct. 17, 2008 (JP) 2008-268679

(51) Int. Cl. A61K 38/21; C07C 275/64; A61K 31/17; A61P 31/14

(52) U.S. Cl. 424/85.4; 564/32; 514/588

(57) ABSTRACT

A novel therapeutic agent for hepatitis C having a high therapeutic effect against hepatitis C is disclosed. The therapeutic agent for hepatitis C contains as an effective ingredient hydroxyurea. The therapeutic agent for hepatitis C optionally further contains interferon α. By the present invention, a novel therapeutic agent for hepatitis C having a high therapeutic effect against hepatitis C was provided. A synergistic therapeutic effect against hepatitis C is obtained by using interferon α in combination. Therefore, in cases where a patient is infected with the genotype of hepatitis C virus for which therapy by interferon α is effective, the therapeutic effect is further promoted by using interferon α in combination.

### TECHNICAL FIELD

The present invention relates to a novel therapeutic agent for hepatitis C.

### BACKGROUND ART

Hepatitis C virus (HCV) is the main causative agent of chronic liver disease which develops into liver cirrhosis and hepatocellular carcinoma. HCV infection is a global health problem with over 170 million people being infected with the virus. HCV genotype 1 is the major genotype found mainly in Japan and the United States, as well as in many other countries. Unfortunately, less than 50% of the patients infected with HCV of this genotype respond to the standard and most effective combination therapy of pegylated interferon and ribavirin. In order to develop a more effective therapy especially for these patients, the present inventors developed a genome-length HCV replication system (Patent Literature 1). By using this assay system, substances having a therapeutic effect against hepatitis C can be screened. Using this assay system, the present inventors found that statins inhibit HCV gene replication (Patent Literature 1). Moreover, Bader et al. conducted a clinical study on the basis of these previous findings and reported that fluvastatin also showed anti-HCV activity in hepatitis C patients (Non-Patent Literature 1).

### PRIOR ART REFERENCES

### Patent Literature

Patent Literature 1: JP 4009732 B

### Non-Patent Literature

Non-Patent Literature 1: T. Bader, J. Fazili, M. Madhoun, C. Aston, D. Hughes, S. Rizvi, K. Seres, M. Hasan, Fluvastatin inhibits hepatitis C replication in humans, Am. J. Gastroenterol. 103 (2008) 1383-1389.)

### DISCLOSURE OF THE INVENTION

### Problems to be Solved by the Invention

As described above, the system for screening a substance having a therapeutic effect against hepatitis C is known as described in Patent Literature 1, and it was found that statins have a therapeutic effect against hepatitis C using this screening. However, the therapeutic effect of statins against hepatitis C is not necessarily satisfactory. Further, even if the above-described screening system is used, it is not easy for finding a substance having a high therapeutic effect against hepatitis C from the infinite number of substances which exist in the world.

An object of the present invention is to provide a novel therapeutic agent for hepatitis C having a high therapeutic effect against hepatitis C.

### Means for Solving the Problems

The present inventors intensively studied using the above-described screening system described in Patent Literature 1 to find that hydroxyurea used as a therapeutic agent for chronic myelogenous leukemia and so on has a high therapeutic effect against hepatitis C, thereby completing the present invention. The present inventors also found that hydroxyurea has a synergistic effect with interferon α on the therapeutic effect against hepatitis C.

That is, the present invention provides a therapeutic agent for hepatitis C, comprising as an effective ingredient hydroxyurea. The present invention also provides a therapeutic agent for hepatitis C according to the therapeutic agent for hepatitis C, further comprising interferon α. The present invention also provides use of hydroxyurea for the production of a therapeutic agent for hepatitis C. The present invention further provides a method of treating hepatitis C, comprising administering to a patient suffering from hepatitis C a therapeutically effective amount of hydroxyurea.

### Effects of the Invention

By the present invention, a novel therapeutic agent having a high therapeutic effect against hepatitis C was provided.

### BRIEF DESCRIPTION OF THE DRAWINGS

FIG. 1A shows the relationship between the culturing time and the relative luciferase activity when various concentrations of hydroxyurea were added to OR6 cells during culturing.

FIG. 1B shows the relationship between the concentration of hydroxyurea and relative luciferase activity when various concentrations of hydroxyurea were added to OR6 cells during culturing.

FIG. 2 shows the relationship between the concentration of hydroxyurea and the relative luciferase activity when OR6c cells which were OR6 cells cured by IFN were transfected with a plasmid encoding Renilla luciferase driven by the internal ribosome entry site (IRES) of encephalomyocarditis virus (EMCV) was transfected into the OR6c cells using FuGENE6 reagent (trade name, produced by Roche Diagnostics), various concentrations of hydroxyurea were added thereto 24 hours after the transfection and the resulting OR6c cells were cultured for 72 hours.

FIG. 3 shows the relative cell number when OR6 cells were cultured in a culture medium to which various concentrations of hydroxyurea were added.

FIG. 4A shows the relationship among the concentrations of hydroxyurea and IFNα, and the relative luciferase activity when various concentrations of hydroxyurea were added to the OR6 cells during culturing.

FIG. 4B shows the results of isobologram analysis of the measurement results shown in FIG. 4A.

### BEST MODE FOR CARRYING OUT THE INVENTION

As described above, the therapeutic agent for hepatitis C of the present invention comprises hydroxyurea (hereinafter also referred to as “HU”) as an effective ingredient. HU per se is a well-known compound having the chemical structure shown below, and its production process is also well-known. HU has long been commercially available as a therapeutic agent for chronic myelogenous leukemia and so on. In the present invention, commercially available HU may conveniently be used.

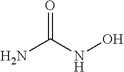

The therapeutic agent for hepatitis C of the present invention may contain other active ingredient(s) in addition to HU. For example, as will be concretely described in the Examples below, it was revealed that HU has an effect to promote the therapeutic effect of interferon α which is conventionally used as a therapeutic agent for hepatitis C, that is, HU has a synergistic effect with interferon a on the therapeutic effect against hepatitis C. Therefore, the therapeutic agent for hepatitis C of the present invention preferably further contains interferon α. As the interferon α used as a pharmaceutical, PEG interferon α is widely used, which is interferon α to which polyethylene glycol chain is added to increase the in vivo stability thereof. The term “interferon α” used in the present Description and Claims is meant to include the stabilized derivatives of interferon α such as PEG interferon α, whose in vivo stability is promoted (except the terms used in the Examples). The other active ingredients which the therapeutic agent for hepatitis C of the present invention optionally contains are not restricted to interferon α, and the therapeutic agent for hepatitis C of the present invention optionally contains, for example, ribavirin used for the therapy of hepatitis C, or optionally contains both of interferon α and ribavirin.

The administration route may be either oral or parenteral. Examples of the parenteral administration routes include, but are not limited to, intravenous administration, intramuscular administration, topical administration to the liver or vicinity thereof, and percutaneous administration. Among these, oral administration is preferred. The dosage form may be any of the known dosage forms, and examples thereof include, but are not limited to, tablets, capsules, lozenges, troches, hard candies, powders, sprays, creams, liniments, suppositories, jellies, gels, pastes, lotions, ointments, aqueous suspensions, injection solutions, elixirs and syrups.

The therapeutic agent for hepatitis C of the present invention optionally contains a vehicle(s), carrier(s), diluent(s) and so on for formulations in addition to HU and, in some cases, the above-described other active components, and usually contains such a component(s) for formulation. These components are well-known in the field of pharmaceuticals, and examples thereof include, but are not limited to, lactose, starch, cellulose, calcium carbonate, sodium alginate and water. Other additives conventionally used in the field of formulation, such as salts for giving buffer action or for adjusting osmosis, solubilizers, dispersing agents, stabilizers, anti-oxidants, preservatives, disintegrating agents, binders, corrigents, lubricants, coating agents, coloring agents and the like may be blended.

The dose may be appropriately selected depending on the conditions, clinical history, severity and body weight of the patient, and other active components to be used in combination, and is usually about 1 mg to 10000 mg, preferably about 500 mg to 2,000 mg in terms of the amount of HU per day per adult. In cases where PEG interferon α is used in combination, the dose of PEG interferon α is also selected similarly, and is usually about 1 μg to 1000 μg, preferably about 10 μg to 200 μg in terms of the amount of PEG interferon α per day per adult. The dosing period may be appropriately selected observing the conditions of the patient, and is usually about 1 day to 1500 days, particularly about 14 days to 730 days.

The therapeutic agent for hepatitis C of the present invention is effective for both of acute hepatitis C and chronic hepatitis C. As will be concretely described in the Examples below, since the therapeutic agent for hepatitis C of the present invention was proved to be effective for the treatment of chronic hepatitis C, particularly intractable chronic hepatitis C, the therapeutic agent of the present invention well exhibits its power when used for the treatment of chronic hepatitis C, particularly intractable chronic hepatitis C. The term “chronic hepatitis C” means the hepatitis caused by hepatitis C virus, which continues for 6 months or more, and the term “intractable hepatitis C” means the hepatitis C patient is infected with hepatitis C virus of genotype 1 wherein the amount of HCV RNA measured by TaqMan (trade name) PCR method is 5 Log IU/ml or more.

As will be concretely described in the Examples below, the concentration of HU for inhibiting replication of hepatitis C (HCV) by 50% in the screening system using the cells described in Patent Literature 1 is 60 μmol/L which is lower than the concentration of ribavirin (76-126 won) that is a commercially available therapeutic drug for hepatitis C. Therefore, the therapeutic agent is thought to have a high therapeutic effect against hepatitis C. The permissible dose of HU in human is set to 800 mg/mm² per oral administration every 4 hours, and in this case, the blood HU level reaches as high as 2480 μmol/L. Therefore, HU is thought to be clinically used individually. Further, as will be concretely described in the Examples below, a synergistic effect on the therapeutic effect against hepatitis C is obtained by using interferon α in combination. Therefore, in cases where the patient is infected with hepatitis C virus whose genotype is that for which the therapy by interferon α is effective, the therapeutic effect is further promoted by using interferon α in combination.

### EXAMPLES

The present invention will now be described more concretely by way of examples thereof. It should be noted, however, that the present invention is not limited to the Examples below.

### Formulation Example 1

As an example of the composition of capsules for oral administration, the following may be exemplified.

| | |
|---|---|
| HU | 500 mg |
| Hydrea capsule (Bristol-Myers) | 500 mg |

### Example 1

### Anti-HCV Activity of HU

The compounds having a therapeutic effect against hepatitis C were screened by the screening system using the cells described in Patent Literature 1 developed by the present inventors.

Briefly, the cells described in Patent Literature 1 used for the screening were as follows: That is, the cells were those obtained by introducing, into Oc cells (FERM P-20517) originated from a human hepatocarcinoma cell line HuH-7, a plasmid containing full length of HCV genome, luciferase gene as a reporter gene and neomycin-resistant gene (neomycin phosphotransferase gene) as a selection marker gene. More specifically, the cells (OR6 cells) were the Oc cells (FERM P-20517) originated from a human hepatocarcinoma cell line HuH-7, into which a plasmid having an RNA containing, from the 5′-end, internal ribosome entry site (IRES) sequence of HCV, luciferase gene, neomycin phosphotransferase gene, IRES sequence originated from encephalomyocarditis virus (EMCV), HCV open reading frame sequence and HCV 3′ untranslated sequence. After culturing the OR6 cells in a culture medium containing a test compound whose anti-viral activity against HCV (that is, therapeutic effect against hepatitis C) is to be examined, the luciferase activity of the cells is measured. The less the replication of the HCV gene, the higher the anti-HCV activity of the test compound, and the lower the luciferase activity. Therefore, the lower the luciferase activity measured, the higher the anti-HCV activity. In Patent Literature 1, it was experimentally confirmed that correlation between the luciferase activity measured and the amount of HCV RNA is very high, so that it has been confirmed that this system has a high reliability.

### 1. Materials and Methods

### (1) Cell Cultures and Antiviral Assays

OR6 cells were cultured in 24-well plates (culture medium: DMEM medium supplemented with 5% fetal calf serum and G418, temperature: 37° C.). In order to monitor the antiviral effect of HU and interferon α (hereinafter referred to as “IFNα”), OR6 cells were plated onto 24-well plates at a density of 15,000 cells per well and cultured for 24 hours. Subsequently, the cells were treated with various concentrations of human IFNα or HU or a combination of the 2 for 72 h. Luciferase activity was assayed using the Renilla luciferase assay system (Promega) by collecting the cells and measuring the luciferase activity in accordance with the instructions attached to the product. Luciferase activity was measured using a manual Monolight 3010 luminometer (BD Biosciences).

### (2) Test Compounds

HU, which was over 98% pure, and human IFNα were purchased from Sigma-Aldrich.

### (3) Cell Viability

In order to examine the cytotoxic effects of HU on OR6 cells, the cells were seeded onto dishes of diameter 95 mm at a density of 400,000 cells per dish, and cultured for 72 hours after adding HU to a concentration of 0, 50, 100, and 150 μmol/L, respectively. The number of viable cells was then counted by a hematocytometer after staining with trypan blue dye.

### (4) Transfection Experiment

In accordance with Patent Literature 1, a plasmid encoding Renilla luciferase driven by the internal ribosome entry site (IRES) of encephalomyocarditis virus (EMCV) was transfected into OR6c cells which were OR6 cells cured by IFN, with FuGENE6 reagent (produced by Roche Diagnostics), and 24 hours after transfection, the cells were treated with various concentrations of HU and cultured for 72 hours.

### 2. Results

### (1) HU Alone Showed Inhibitory Effects on HCV Gene Replication

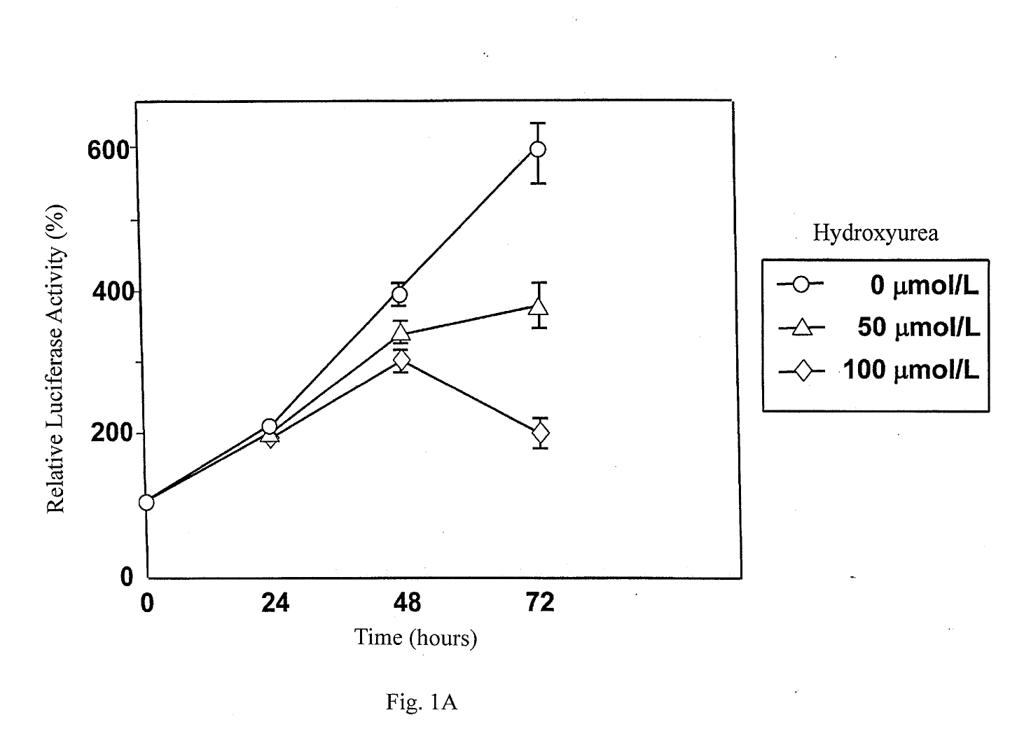

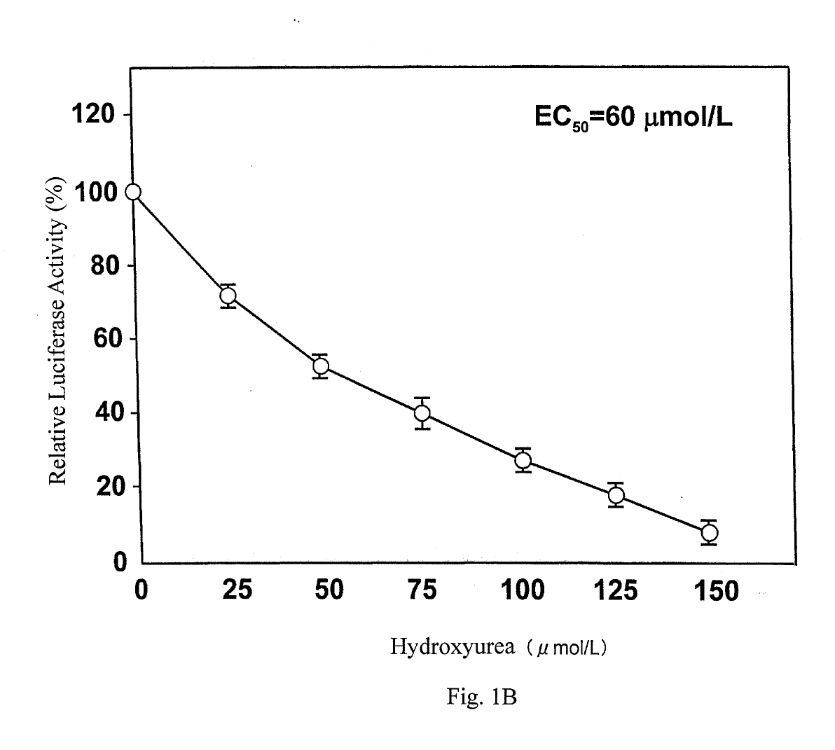

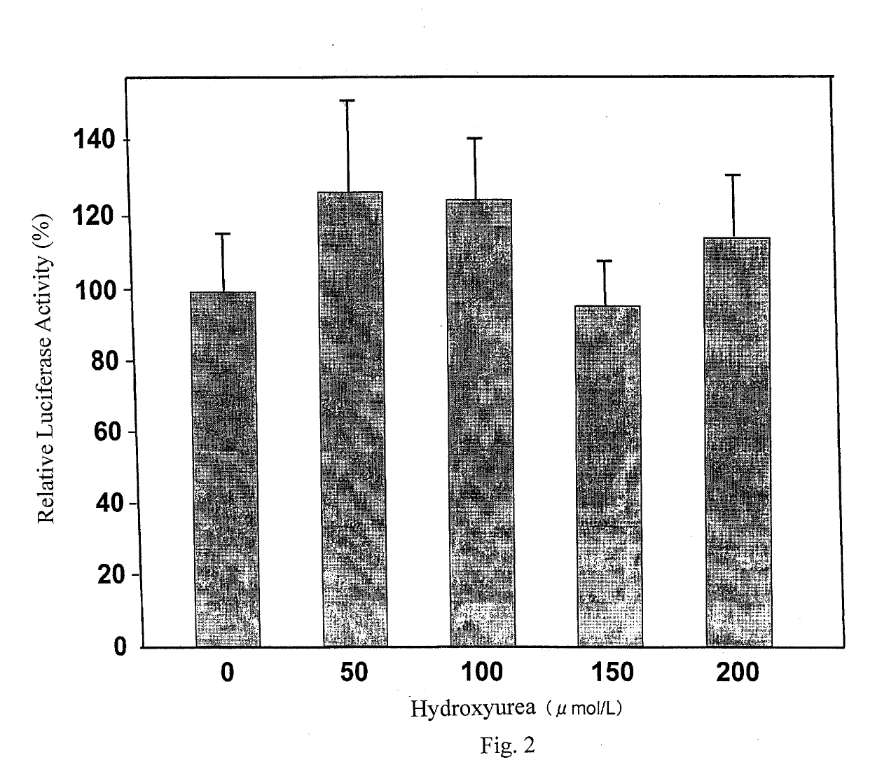

Since OR6 cells are considered to be a reliable system for monitoring HCV RNA replication, it was evaluated whether HU alone could inhibit the replication of genome-length HCV gene in OR6 cells (FIG. 1A). According to the dose-response curve obtained after 72-h treatment with HU up to 150 μmol/L, the 50% replication inhibition concentration (the concentration at which the replication is inhibited by 50% when compared to the case where HU was not added) of HU was estimated as 60 μmol/L (FIG. 1B). The 50% inhibition concentration of interferon α was estimated as 1.2 IU/mL. It was confirmed that HU did not inhibit Renilla fluorescence by using cured OR6c cells transfected with a control plasmid pEMCV-RL (FIG. 2). These results revealed that HU can independently inhibit HCV gene replication.

### (2) The Anti-HCV Activity of Hu was not Due to Cytotoxicity

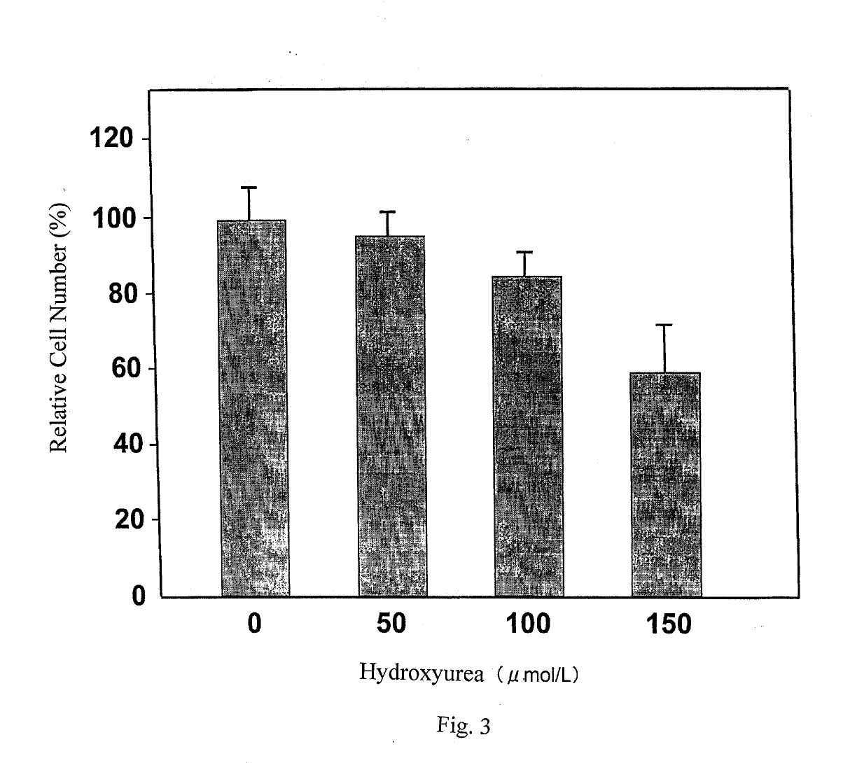

Since it has been reported that the proliferation of the HCV replicon is dependent on host-cell growth, there is a possibility that the inhibitory effects of HU on HCV gene replication are attributable to its cytotoxic effects. To eliminate this possibility, the cytotoxic effects of HU on OR6 cells were investigated. As a result, no significant difference was observed in the number of viable cells in the samples treated with HU at each concentration up to 100 μmol/L as compared to the number in the untreated cells (FIG. 3).

### (3) Combination of HU and IFNα Effectively Inhibited HCV Gene Replication

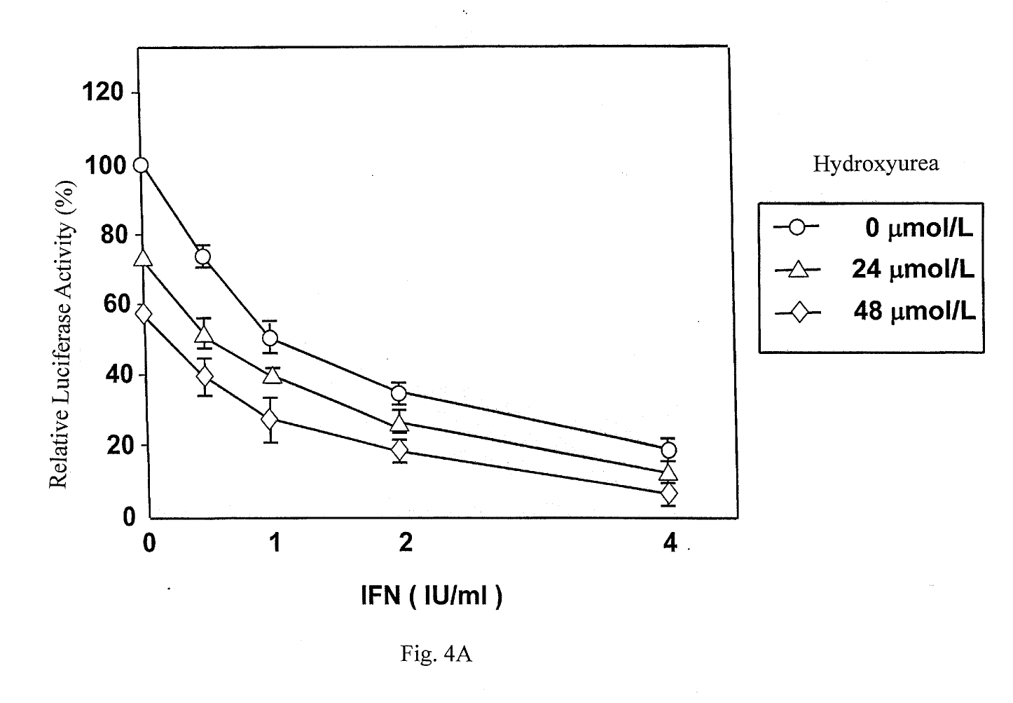

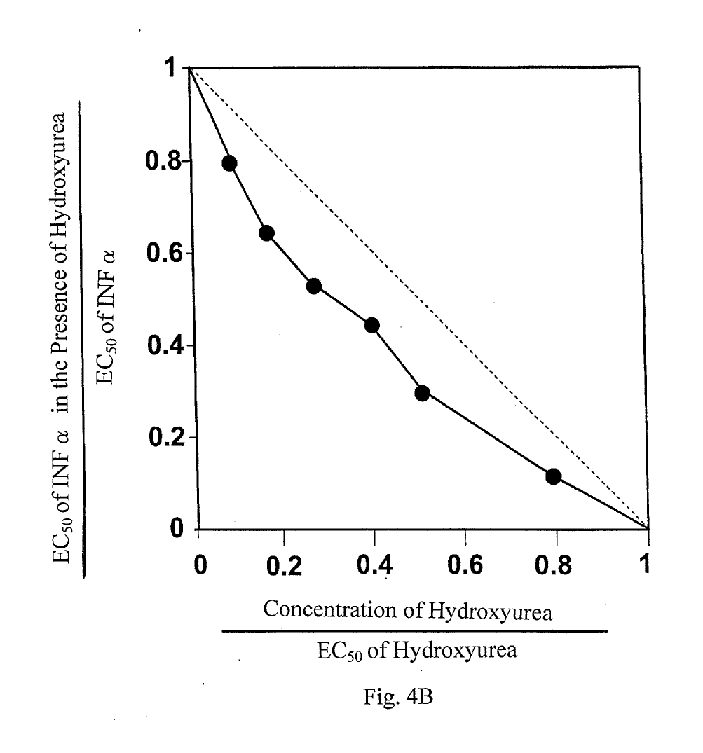

Next, the inhibitory effects of the combination of IFNα and HU on genome-length HCV RNA replication were investigated. The dose-response curves of IFNα were obtained for each of the following fixed concentrations of HU: 0, 24, and 48 μmol/L. The curves shifted to the left with increasing concentrations of HU (FIG. 4A), indicating that co-treatment was more effective than treatment with IFNα alone. An isobologram revealed the synergistic anti-HCV activity of the IFNα and HU combination (FIG. 4B. In FIG. 4B, the broken line indicates the case where there is no synergistic effect (additive effect), and the fact that the found values are in the left lower side thereof indicates that anti-HCV effect is higher by the combination of HU and IFNα than by IFNα alone). These results revealed that HU can be used as an anti-HCV reagent in combination with IFNα.

### Example 2

### Clinical Tests

Hydroxyurea (trade name: Hydrea Capsule) was orally administered to 12 patients suffering from intractable hepatitis C (serogroup 1, high virus level), who gave informed consent, at a dose of 1500 mg/day for 4 weeks. Serum HCV RNA level (log IU/ml) was measured every week from before the administration to the end of the therapy by the real-time detection PCR method using TaqMan (trade name) probe.

As a result, the decrease in the virus level (HCV RNA) was observed in 9 patients (75%) out of 12 patients. The decrease in the virus level was 0.37 Log at maximum and 0.22 Log on average.

### INDUSTRIAL APPLICABILITY

By the present invention, a novel therapeutic agent for hepatitis C useful for the treatment of hepatitis C patients was provided. The therapeutic agent of the present invention is also useful for the treatment of intractable chronic hepatitis C, so that it will contribute to the therapy of hepatitis C.

1. A therapeutic agent for hepatitis C, comprising as an effective ingredient hydroxyurea.

2. The therapeutic agent according to claim 1, further comprising interferon α.

3. Use of hydroxyurea for the production of a therapeutic agent for hepatitis C.

4. A method of treating hepatitis C, comprising administering to a patient suffering from hepatitis C a therapeutically effective amount of hydroxyurea.

5. The method according to claim 1, further comprising administering, in combination, interferon α in an amount further promoting therapeutic effect.
